(19) United States
(12) Patent Application Publication
(10) Pub. No.: US 2010/0228333 A1
(43) Pub. Date: Sep. 9, 2010

(54) Drug eluting surface covering

(76) Inventors: William Joseph Drasler, Minnetonka, MN (US); Joseph Michael Thieten, Buffalo, MN (US)

Correspondence Address: William J. Drasler, 4100 Dynasty Drive, Minnetonka, MN, 55345, US

(21) Appl. No.: 12/660,711

(22) Filed: Mar. 3, 2010

Related U.S. Application Data
(60) Provisional application No. 61/209,144, filed on Mar. 4, 2009.

(51) Int. Cl. A61L 31/16; A61M 25/10; A61F 2/84

(52) U.S. Cl. 623/1.11; 604/103.02; 604/103.05

(57) ABSTRACT

A thin-walled sheath is placed over a balloon having an antirestenotic drug placed on the balloon of a balloon dilatation catheter. The sheath protects the drug from dissolution into the blood and allows improved delivery to the lesion site. A rolling action of the sheath prevents the drug from loss due to shearing motion. The sheath can also provide a protected surface for carrying the drug and providing exposure to the lesion site for delivery of the drug. The sheath can also serve as a delivery sheath for providing delivery of a stent via a single catheter introduction for drug delivery and stent delivery.

### CROSS REFERENCE TO RELATED APPLICATIONS

This invention makes reference and thereby includes aspects of the provisional patent application entitled Drug Eluting Surface Covering with application No. 61/209,144 filed on 04 Mar. 2009 by William J. Drasler and Joseph M. Thielen.

### BACKGROUND OF THE INVENTION

1. Field of Invention

This invention relates to interventional devices used to percutaneously enter into blood vessels, dilate the vessel, and provide a drug that helps to prevent the restenosis of the blood vessel. It could involve angioplasty balloon catheters, stents, and drugs such as paclitaxel, sirolimus, and others to assist in reducing the tendency toward inflammation, thrombosis, cellular proliferation, and other mechanisms leading to restenosis.

2. Description of Prior Art

Balloon angioplasty has provided an option to surgery for the treatment of diffuse and focal lesions in blood vessels of the body. Although the results were of great benefit to many patients, the restenosis rates were reasonably high with restenosis of the coronary arteries at approximately thirty percent. The restenosis was due to inflammation and hyperplastic response due to the vessel injury. Bare metal stents were then used along with balloon angioplasty to reduce recoil of the vessel and provide a larger lumen size. The restenosis rates were significantly better although restenosis rates remained at high levels. Drugs such as paclitaxel and sirolimus were placed on the stents to provide local inhibition of cellular hyperplasia resulting in single digit rates for restenosis. However, long term healing of the stent did not occur due to presence of the drug over a longer period of time. As a result, some stents tended to cause thrombosis at later time periods, resulting in a small but significant myocardial infarction rate that could lead to death in some patients.

Studies have been conducted to examine the potential benefit of placing drug directly onto the balloon and forcing the balloon into contact with the vessel wall during a balloon angioplasty procedure. A bare metal stent can be implanted following use of a drug eluting balloon to reduce recoil of the vessel. Since the drug is not being eluted from a stent over a long period of time, the chances of late vessel thrombosis have been significantly mitigated. The results have demonstrated that short term application of the drug via a balloon does reduce restenosis by reducing cellular hyperplasia.

Since the angioplasty balloon is in contact with the vessel wall for only a brief period of time, i.e., 30-60 seconds, it is important to place additives along with the drug to enhance their uptake into the vessel in a short period of time. Many molecular species have been examined as potential additives to the therapeutic drug agent to form drug mixtures. Additives include contrast agents, surfactants and other molecules with both hydrophilic and hydrophobic moieties, and substances that enhance the penetration of the drug into the vessel wall such as dimethylsulfoxide, and others.

It is important to prevent the drug mixture from washing off of the balloon while it is being delivered to the lesion site. Various adhesive layers have been examined to help improve the bonding of the active drug agent or drug mixture to the outside of the balloon. One difficulty however is to provide rapid release of the drug to the vessel wall at the lesion site but not to loose the drug to the blood or the vessel wall during its delivery path to the lesion site.

One method that is commonly used to improve the delivery of the drug to the vessel site is to wrap the wings of the deflated balloon around the drug. This method provides some benefit to protect the drug but only can protect approximately 50-60 percent of the outer surface of the deflated balloon. Also the wrapped balloon does not provide an even distribution of the drug along the entire perimeter of the balloon if some of the drug has been washed off by exposure to blood or abraded off. An improved device is needed to protect the drug from loss during its delivery and allow the drug to be delivered uniformly to the vessel lesion.

### SUMMARY

The present invention is an external sheath having a thin wall and being placed over a balloon angioplasty catheter to protect a restenosis drug or drug mixture from being washed away during delivery. The drug mixture can be placed onto the balloon of the balloon catheter or it can be placed on the sheath. The sheath is inverted upon itself such that as the sheath is removed from covering the drug mixture, it does not cause the drug mixture to be sheared off. The drug mixture can be bonded to a surface with an adherent layer.

In one embodiment a drug mixture is located on the outside surface of the balloon and a sheath of the present invention covers the drug located on the balloon. The drug mixture can contain an active therapeutic agent, an additive and a contrast agent or it can contain one of two of these substances. The drug mixture could also contain another substance that provides a benefit if it is delivered to a vessel wall. An adherent layer can be used to bond the drug mixture and other molecules to the surface of the balloon or to other surfaces as described in other embodiments. The active agent can be paclitaxel and analogues thereof, rapimycin and analogues thereof including sirolimus, paclitaxel, or other identified anti-inflammatory, antiproliferative, antithrombotic, anticancer, or other drug that helps reduce the restenosis, inflammation, thfOmbosis, or provide a physiological benefit to the vessel following angioplasty or stenting. Some additives that enhance the penetration of the drug into the vessel wall can' include surfactants that are ionic, nonionic, zwitterionic, and other molecules with hydrophilic and hydrophobic moieties. Some solvents such a dimethylsulfoxide and others can improve the penetration into the vessel wall. Various contrast agents both ionic and nonionic can also be used in the drug mixture.

In this embodiment a thin-walled sheath extends along the angioplasty catheter and covers the drug mixture located on the outside surface of the balloon. The sheath is inverted in its distal portion such that it unwraps with a motion similar to a tank tread and does not cause the drug mixture to be sheared off. The sheath can be a thin-walled elastomeric member formed of elastic materials such as silicone, polyurethane, a copolymer of these, or other elastic material. The sheath can also be formed of a non-compliant or semi-compliant material such as polyethylene terephthalate, polyvinylchloride, polyethylene, pebax and others commonly used in dilatation balloons and interventional catheters. During delivery of the balloon catheter to the lesion site, the sheath covers the drug mixture and protects it from exposure to the blood and contact with the vessel wall that could cause the drug mixture to be sheared off. Once the balloon catheter and sheath are delivered to the site of the lesion, the sheath is retracted to expose the drug mixture that is located on the balloon to the blood. The balloon on the balloon angioplasty catheter is inflated to place the drug mixture into contact with the vessel wall. The distal end of the sheath can be attached near the proximal end of the balloon to ensure that the drug mixture on the sheath is positioned properly over the balloon during balloon inflation.

In another embodiment, the drug mixture is again located on the surface of the balloon. The sheath is inverted in a distal portion and positioned over the drug mixture to protect it from blood exposure or vessel contact during delivery. In this embodiment the distal end of the sheath is not attached to the balloon catheter thereby allowing the sheath to be used as an annular conduit after the sheath has been retracted.

In another embodiment, the drug mixture is located on the sheath. The sheath is positioned over the balloon catheter and the distal portion of the sheath is inverted such that a portion of the outside surface of the sheath becomes an inwardly facing surface that faces the balloon catheter. The drug mixture is placed onto this inwardly facing surface. The distal end of the sheath can be attached to the proximal end of the balloon. The balloon catheter with the sheath covering can be delivered to the site of the lesion with the drug mixture protected from blood and contact with the vessel wall. The catheter is delivered to a position within the vessel such that the drug mixture can be placed into contact with the lesion. Retraction of the sheath in a proximal direction causes the sheath to form a tank tread motion and place the drug above the balloon and facing the blood vessel at the site of the lesion. Inflation of the balloon will inflate the sheath having the drug mixture attached and pushes the drug mixture into contact with the lesion of the blood vessel. The sheath can be made of an elastomeric material such as silicone, polyurethane, or other commonly used elastomeric and can stretch during the inflation of the balloon. Alternately, the sheath can be formed of a non-compliant or semi-compliant material that is folded in its delivery conformation and unfolded during the balloon inflation.

In another embodiment the drug mixture is attached to the sheath as discussed in the last embodiment but the sheath is not attached to the balloon. Following inflation of the balloon and delivery of the drug mixture to the lesion of the vessel wall, the sheath can remain in place while the balloon is deflated. The balloon catheter can be removed with the sheath remaining in place if desired. The sheath can be used as a conduit for delivery of devices or liquids to the vasculature.

In another embodiment, the drug mixture is attached to the sheath as discussed in the last embodiment but the sheath can have the inverted portion extend throughout approximately the entire length of the sheath. Exposure of the drug mixture located on the sheath to the blood can be attained by retraction of the proximal end of the sheath. Inflation of the balloon then can place the drug mixture into contact with the blood vessel wall. Retraction of the distal end of the sheath then allows the sheath to be peeled away from the vessel wall without exposing the drug mixture to a shearing motion that could cause the drug mixture to be unnecessarily stripped off by the vessel wall.

In an additional embodiment the thin-walled sheath is placed over the balloon that has a therapeutic drug coated onto its outer surface. In this embodiment the sheath is not inverted in its distal portion. The balloon catheter and sheath are delivered to the lesion site with the sheath protecting the drug coated balloon from exposure to blood. Retraction of the sheath in a proximal direction exposes the drug coated balloon to the blood and the lesion site. The balloon can be inflated to place the drug into contact with the vessel wall.

In a further embodiment the sheath of the present invention is positioned over a balloon catheter and used to hold a self-expanding stent in a small diameter state as well as provide protection to a drug mixture during the delivery of the sheath to the lesion site. The drug mixture can be located on the outer surface of the balloon and the stent can be positioned on the balloon catheter in a location proximal to the balloon during delivery. Proximal retraction of the sheath having an inverted wall in its distal portion can expose the drug mixture to the vessel wall and allow for expansion of the balloon to place the drug into contact with the vessel wall. Further retraction of the sheath allows the self-expanding stent to be released to the lesion site. The balloon can be used to post dilate the self-expanding stent if desired.

In an alternate embodiment the inverted sheath positioned over a balloon catheter is used to deliver a self-expanding stent and provide protection to the drug mixture that is located on the inverted portion of the sheath. Proximal retraction of the sheath exposes the drug mixture to the lesion site and places the drug directly over the balloon. Expansion of the balloon forces the drug mixture against the vessel wall. Further proximal retraction of the sheath releases the self-expanding stent into the vessel at the lesion site.

In yet another embodiment, the inverted sheath is used to deliver a balloon-expandable stent to the lesion site as well as protect a drug mixture located on the outer surface of the balloon. The balloon-expandable stent can be located on the balloon catheter at a location proximal to the balloon. Proximal retraction of the proximal end of the sheath exposes the drug mixture and allows the balloon to be expanded to place the drug into contact with the vessel wall. Proximal movement of the balloon catheter places the main dilatation balloon inside of the stent and a small inflation of the balloon fixes the balloon onto the stent. Proximal retraction of the sheath exposes the balloon-expandable stent loaded onto the balloon to the blood vessel and is available for balloon inflation at the site of the lesion. A proximal balloon portion to the balloon or a second balloon can be used to predilate the balloon-expandable stent prior to retracting the main dilatation balloon inside of the stent.

In another embodiment, the inverted sheath is used to deliver a balloon-expandable stent and provide protection to a drug mixture located on the sheath. The balloon-expandable stent can be located on the balloon catheter shaft (85) proximal to the balloon. Proximal retraction of the sheath exposes drug located on the inwardly-facing surface of the sheath to the blood vessel and positions it directly above the balloon. Expansion of the balloon and the sheath places the drug mixture into contact with the vessel wall. Movement of the balloon catheter in a proximal direction places the balloon within the stent. Partial inflation of the balloon fixes the balloon to the stent. Proximal retraction of the sheath exposes the stent to the blood vessel and allows the balloon to be inflated to place the stent into contact with the lesion.

### BRIEF DESCRIPTION OF THE DRAWINGS

FIG. 1A is a lateral sectional view of a sheath surrounding a balloon dilatation catheter having drug located on the balloon and covered by the sheath.

FIG. 1B is a lateral sectional view of a sheath surrounding a portion of a balloon dilatation catheter with the sheath retracted and exposing the drug located on the outside surface of the balloon.

FIG. 1C is a cross sectional view of balloon and sheath shown in FIG. 1A.

FIG. 1D is a cross sectional view of a balloon having drug located on its outside surface.

FIG. 2A is a lateral sectional view of a balloon dilatation catheter with a sheath surrounding the balloon.

FIG. 2B is a lateral sectional view of a balloon dilatation catheter with a sheath retracted away from the balloon.

FIG. 2C is a lateral sectional view of a balloon dilatation catheter with a sheath surrounding the balloon and not attached to the balloon dilatation catheter.

FIG. 3A is a lateral sectional view of a balloon dilatation catheter with a sheath surrounding the balloon and having drug attached and protected.

FIG. 3B is a lateral sectional view of a balloon dilatation catheter with a sheath retracted exposing drug outwards with the balloon non-inflated.

FIG. 3C is a lateral sectional view of a balloon dilatation catheter with a sheath surrounding the balloon and retracted with the balloon expanded.

FIG. 3D is a cross-sectional view through the nose of balloon catheter of FIG. 3A with balloon non-inflated.

FIG. 3E is a cross-sectional view through the balloon of FIG. 3B with balloon non-inflatated and sheath being semi-compliant.

FIG. 3F is a cross-sectional view through the balloon of FIG. 3C with balloon inflatated and sheath being expanded.

FIG. 3G is a cross-sectional view through the balloon of FIG. 3B with balloon non-inflatated and sheath being non-compliant.

FIG. 4A is a lateral sectional view of a balloon dilatation catheter with a sheath surrounding the balloon and drug located on the sheath and protected.

FIG. 4B is a lateral sectional view of a balloon dilatation catheter with a sheath surrounding the balloon and drug located on the sheath and exposed.

FIG. 5 is a lateral sectional view of a balloon dilatation catheter with a sheath surrounding the balloon and sheath having both ends extending over the shaft of the balloon catheter.

FIG. 6 is a lateral sectional view of a balloon dilatation catheter with a sheath surrounding the balloon and drug located on the balloon.

FIG. 7 is a lateral sectional view of a balloon dilatation catheter with a sheath surrounding the balloon having drug attached and a self-expanding stent located on the balloon catheter shaft.

FIG. 8 is a lateral sectional view of a balloon dilatation catheter with a sheath having drug attached surrounding the balloon and having a self-expanding stent located on the balloon shaft.

FIG. 9A is a lateral sectional view of a balloon dilatation catheter with a sheath surrounding the balloon having drug attached and a balloon-expandable stent located on the balloon catheter shaft.

FIG. 9B is a lateral sectional view of a balloon dilatation catheter with a sheath surrounding the balloon having drug attached and a balloon-expandable stent located on a proximal portion of a balloon located on the balloon catheter shaft.

FIG. 10 is a lateral sectional view of a balloon dilatation catheter with a sheath having drug attached surrounding the balloon and having a balloon-expandable stent located on the balloon catheter shaft.

### DETAILED DESCRIPTION OF THE INVENTION

The present invention is a sheath that provides protected delivery of a drug mixture to the site of a lesion. The drug mixture is protected from the blood which can cause a portion to become diffused away or dissolved during delivery of a catheter containing the drug to the lesion site. The drug mixture can be located on the outside of a balloon of a balloon catheter and can be protected by the sheath of the present invention that is located over the balloon catheter and over the drug located on the balloon. Alternately, the drug mixture can be located on the sheath which protects the drug mixture during delivery and provides exposure of the drug mixture to the vessel wall after the catheter is located properly to provide therapy to the lesion located in the vessel. The balloon catheter provides the outward force that places the drug mixture into contact with the vessel wall.

The drug mixture can contain an active therapeutic agent such as paclitaxel or analogues thereof, rapimysin or analogues thereof including sirolimus, everolimus, and others, or other anti-proliferative drug, anti-cancer drug, anti-inflammatory drug, anti-thrombotic drug, or other drug that provides improved therapeutic results to the vessel following balloon angioplasty, stenting, or other interventional procedures. The drug can be found in the form of micelles or liposomes or microspheres. The therapeutic drug can include a variety of vitamins. The drug can be lipophilic or hydrophilic. The drug mixture can also contain an additive that helps to allow the therapeutic agent to penetrate or permeate the blood vessel wall in a more rapid manner; the additive can also help to improve absorption of the therapeutic drug into tissue and can also improve the release of the therapeutic drug off of the surface of the device. Such additives can have a hydrophilic part and a drug affinity part. Such additives can include surfactants that are ionic, non-ionic, zwitterionic, and other surfactant molecules other molecules and moieties used in drug delivery. The additive can diffuse quickly form the surface of the device carrying the drug along with it. It can be soluble in aqueous or organic solvents. The additives can include various forms of soaps and vitamins. The drug mixture can also contain any type of contrast agent whether it is ionic, nonionic, used for visualization, or used for other purposes. The drug mixture can be placed onto the surface of the balloon or the sheath with an adherent layer to help bond or improve the adherence of the drug mixture to the surface of the device. A top layer can also be placed on top of the drug layer to reduce loss of the drug during delivery and control the release of the drug from the drug layer. The top layer can have a slower release rate if it has a generally greater hydrophobic character although it can also contain hydrophilic groups. Also, a solvent such as dimethylsulfoxide can be added to the drug mixture or placed into a top layer to enhance penetration of the drug into the blood vessel. The drug mixture can be located in a single layer or it can be located in several layers. For example, the therapeutic drug can be found in one layer and the additive and contrast agent can be found in another layer.

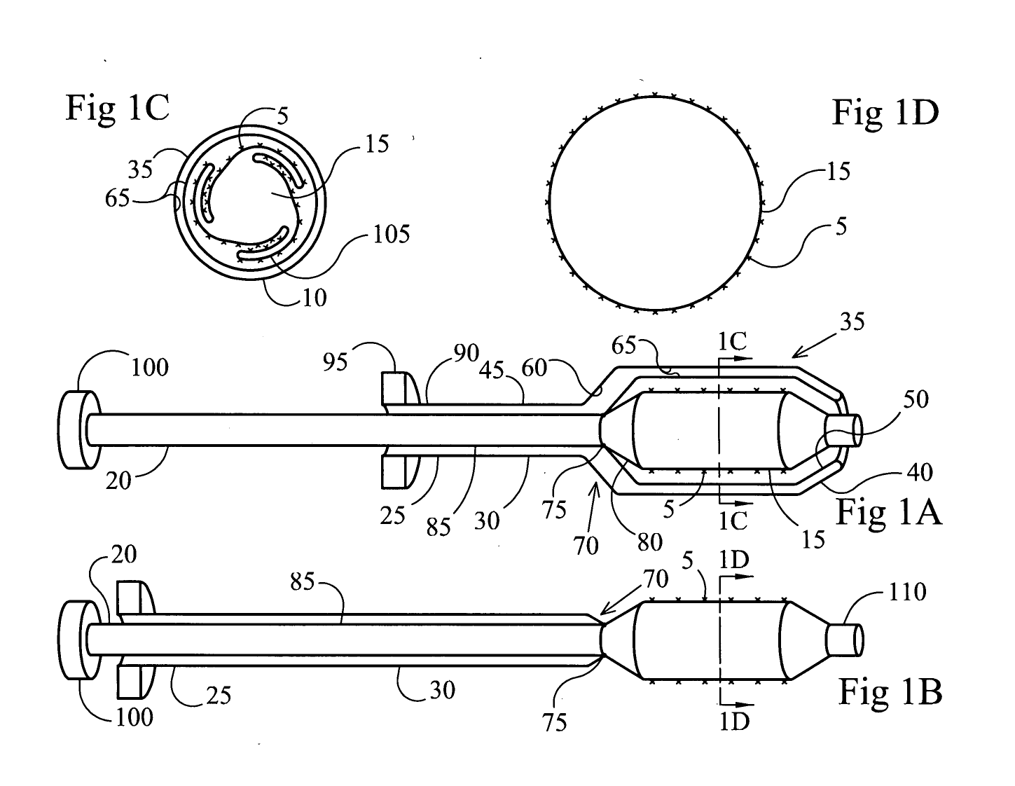

In one embodiment as shown in FIGS. 1A and 1B the drug mixture (5) is located on the outside surface (10) of a balloon (15) of an angioplasty balloon catheter (20). The sheath (25) is a thin walled tubular member (30) that is positioned over the balloon catheter (20). The sheath distal portion (35) is inverted forming an inverted wall (40) such that a portion of the outer surface (45) of the sheath (25) becomes the inwardly facing surface (50) that faces the distal segment (55) of the balloon catheter (20). A portion of the sheath inner surface (60) that faces the balloon catheter (20) becomes an isolated inner surface (65) in which two portions of inner surface (60) face each other. The sheath distal end (70) in this embodiment is attached to the balloon catheter (20) at an attachment site (75). This site can be located on or near the balloon proximal end (80) on the balloon catheter shaft (85). The sheath proximal end (90) is intended to be located outside of the patient's body during the interventional procedure. A control element (95) can be attached to the sheath proximal end (90) to control the movement of the sheath (25) relative to the manifold (100) located on the balloon catheter (20). The control element (95) can be formed with a split design such that it can be removed from the balloon catheter (20) while leaving the balloon catheter (20) in place. The sheath (25) also can be formed such that it can be split axially along its length such that it can be removed while leaving the balloon catheter (20) in place.

The sheath distal portion (35) surrounds the drug mixture (5) which is located on the balloon (15) as shown in FIGS. 1A and 1C. The balloon (15) of the balloon catheter (20) is typically folded to form wings (105) with the drug located on the balloon outside surface (10). The sheath (25) can be formed of an elastic material such as silicone, polyurethane, a copolymer of these, or other elastomeric material commonly used in interventional catheters. The sheath (25) can also be formed of a non-compliant or semi-compliant material such a polyethylene terephthalate, pebax, polyvinylchloride, polyethylene, and other commonly used materials used in making dilatation balloons or interventional catheters. Following balloon inflation as shown in FIG. 1D the drug mixture (5) is placed into contact with the blood vessel.

To use this device the balloon catheter (20) in a deflated condition has the sheath (25) covering the drug mixture (5) located on the balloon (15) as shown in FIG. 1A. The catheter and sheath (25) are advanced into the blood vessel over a guidewire and advanced to the lesion to be treated. The sheath (25) protects the drug mixture (5) from exposure to blood and from direct contact of the balloon (15) with the vessel wall during delivery. Once the catheter is positioned properly with respect to the lesion, the sheath (25) is retracted to expose the vessel wall to the drug as shown in FIG. 1B. Removal of the sheath (25) is with a rolling tank tread type of motion thus not exposing the drug mixture (5) to shear stresses that could cause the drug mixture (5) to be sheared off. The balloon catheter (20) can be advanced distally with respect to the sheath (25) to expose the drug mixture (5) coating on the balloon (15) to the blood and the vessel wall. The balloon (15) can be inflated to place the drug into direct contact with the vessel wall. In one method of use the sheath (25) is withdrawn while holding the balloon catheter (20) stationary to expose the drug mixture (5) to the blood vessel. In an alternative method, the balloon catheter (20) can be advanced while holding the sheath (25) stationary.

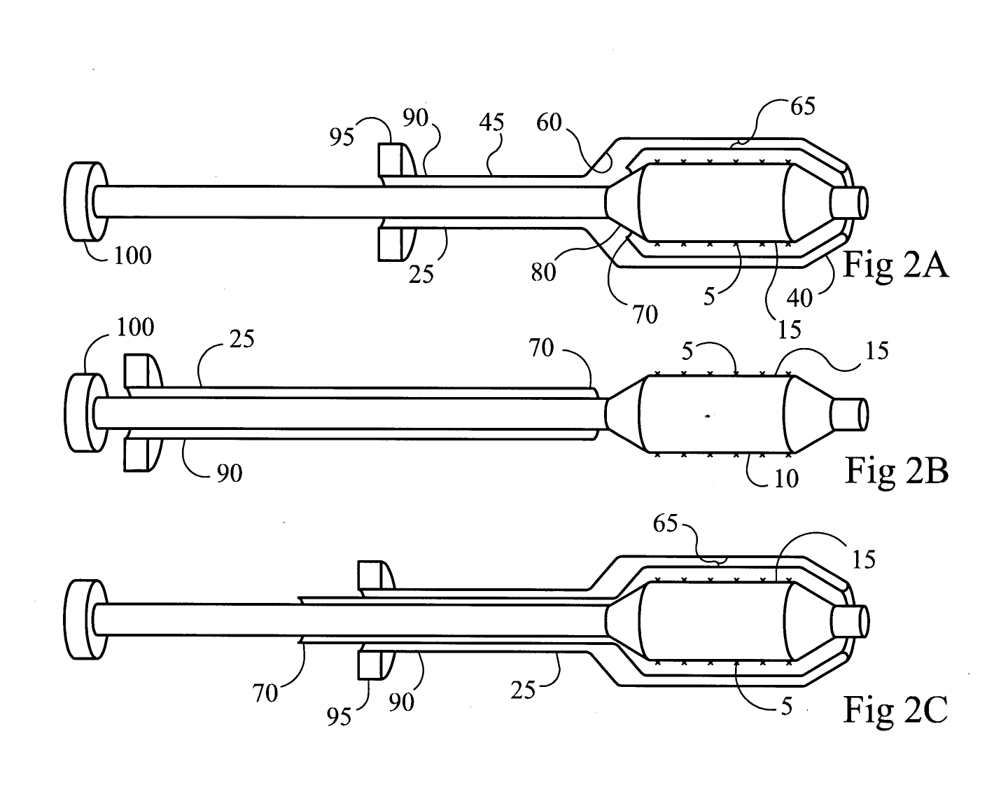

In another embodiment as shown in FIGS. 2A and 2B the drug is located on the balloon outside surface (10) found on the balloon catheter (20). This embodiment is similar to that shown in FIGS. 1A and 1B except that the sheath distal end (70) is not attached to the balloon catheter (20). The sheath (25) is retracted in a manner similar to that of the previous embodiment by distal advancement of the balloon catheter (20) or by retracting the sheath (25) proximally. After exposure of the drug mixture (5) to the vessel as shown in FIG. 2B, the sheath (25) can be removed entirely from the body. A splitting of the sheath (25) along its axial length can allow removal of the sheath (25) over the manifold (100) of the balloon catheter (20). Alternately, the sheath (25) can remain over the balloon catheter (20) and can be used for a conduit for perfusion of fluids if desired.

The embodiment shown in FIG. 2C is similar to that shown in FIGS. 2A and 2B except that the sheath distal end (70) is located along with the proximal end (90) outside of the patient's body. The entire length of the sheath (25) is inverted in this example forming a long isolated inner surface (65). The drug mixture (5) is exposed by retracting proximally the sheath proximal end (90) or by advancing distally the balloon catheter (20) while maintaining the position of the sheath (25).

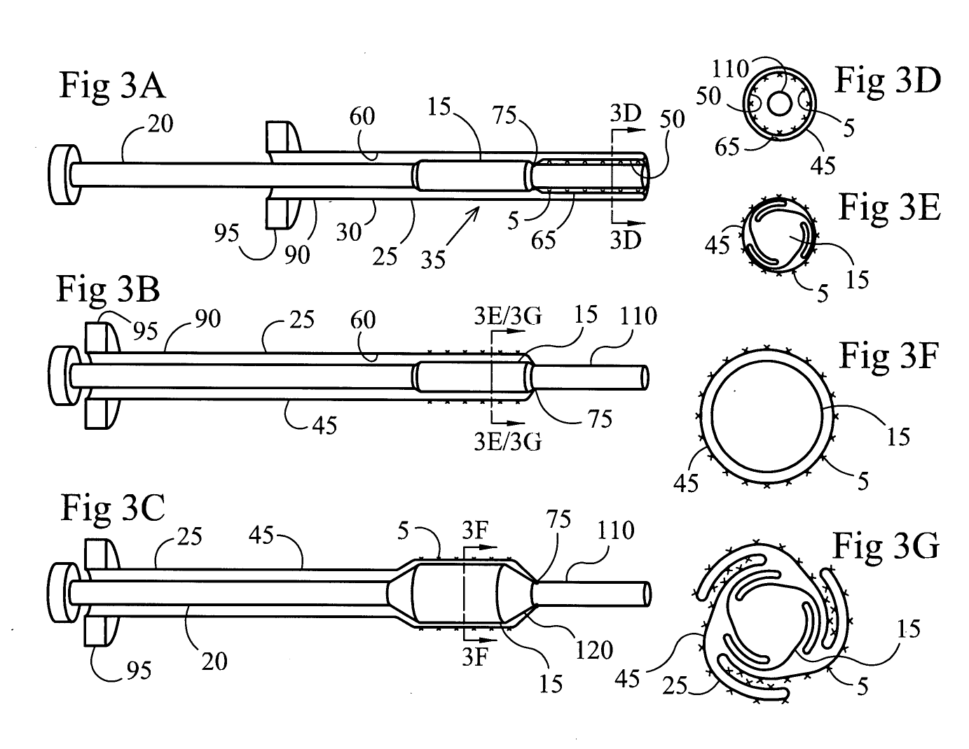

In a further embodiment shown in FIGS. 3A-3C the drug mixture (5) is located on the sheath (25). The tubular member (30) of the sheath (25) is positioned over the balloon catheter (20) including the distal nose (110) of the balloon catheter (20). The sheath distal portion (35) is inverted upon itself such that a portion of the sheath outer surface (45) becomes an inwardly facing surface (50) that faces the distal segment (55) of the balloon catheter (20) including the distal nose (110) of the balloon catheter (20). A drug mixture (5) is placed on the inwardly facing surface (50) of the sheath (25). An adherent layer can also be used to enhance the bonding to the sheath (25). The sheath distal end (70) can be attached to the balloon catheter (20) at an attachment site (75) which can be located near the balloon distal end (120) or on the nose (110) of the balloon catheter (20).

Retraction of the sheath proximal end (90) or distal advancement of the balloon catheter (20) causes the distal portion (35) to unwrap like a tank tread placing the drug mixture (5) onto the sheath outer surface (45) that lies directly over the balloon (15) as shown in FIG. 3B. Inflation of the balloon (15) as shown in FIG. 3C causes the balloon (15) and the sheath (25) to expand and force the drug mixture (5) into contact with the vessel wall.

The sheath (25) can be formed of an elastic material that contains the drug mixture (5) on its inwardly facing surface (50) as shown in FIGS. 3A and 3D. As the sheath (25) is retracted proximally as shown in FIGS. 3B and 3E, the elastic sheath (25) with the drug mixture (5) facing outwards covers the folded balloon (15). Expansion of the balloon (15) causes the sheath (25) to expand outwards as shown in FIGS. 3C and 3F and placing the drug into a position that would come into contact with a blood vessel wall for delivery of the drug to the lesion site.

Alternately, the sheath (25) can be formed out of a non-compliant or semi-compliant material that is folded in its distal portion (35) and placed over the distal segment (55) of the balloon catheter (20) as shown in FIG. 3A. The drug mixture (5) is located on the inwardly facing surface (50) of the sheath distal portion (35). As the sheath (25) is retracted proximally as shown in FIGS. 3B and 3G, the folded non-compliant or semi-compliant sheath (25) with the drug mixture (5) facing outwards on the sheath outer surface (45) covers the folded balloon (15). Expansion of the balloon (15) causes the sheath (25) to unfold outwards and placing the drug mixture (5) located on the sheath outer surface (45) into a position that would come into contact with a blood vessel wall for delivery of the drug mixture (5) to the lesion site.

To use the present embodiment, the sheath (25) and balloon catheter (20) are advanced to the site of the lesion with the balloon (15) nondeployed as shown in FIG. 3A. Once the balloon (15) and sheath (25) are positioned properly to deliver drug to the site of the lesion, the sheath (25) is retracted while holding the balloon catheter (20) stationary or the balloon (15) is advanced while holding the sheath (25) stationary to expose the drug mixture (5) to the vessel wall as shown in FIG. 3B. The balloon (15) is inflated to cause both the balloon (15) and the sheath (25) to expand or unfold as shown in FIG. 3C and cause the drug mixture (5) to come into contact with the vessel wall.

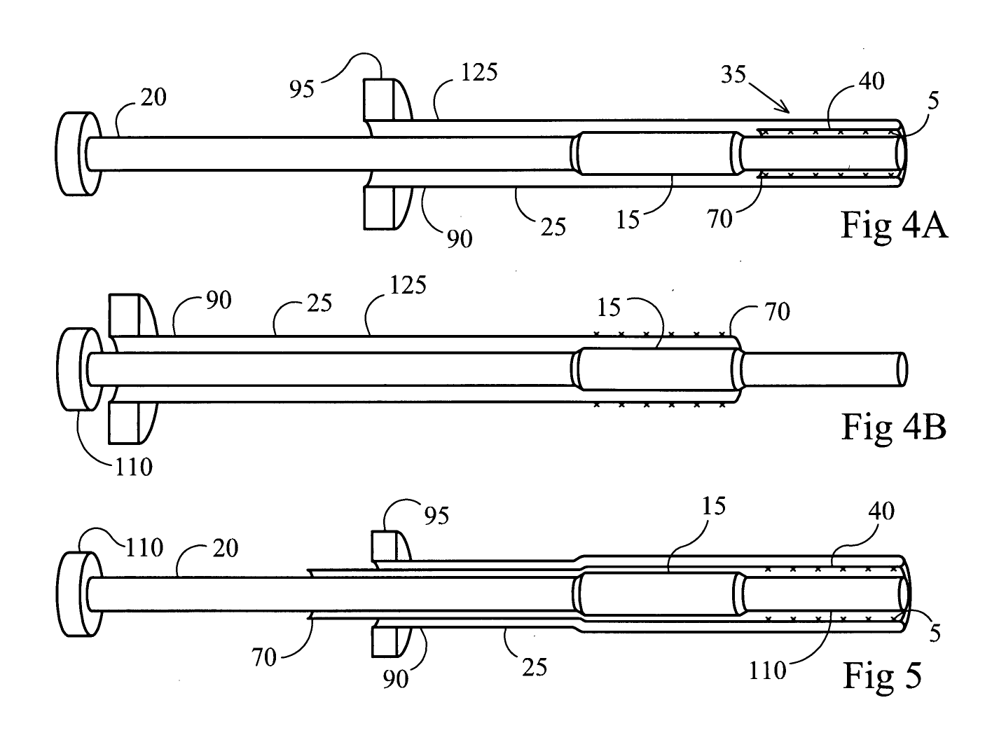

The embodiment shown in FIGS. 4A and 4B are similar to that shown in FIGS. 3A and 3B except that the sheath distal end (70) is not attached to the balloon catheter (20). The lack of attachment allows the balloon catheter (20) to be removed following inflation thereby leaving the sheath (25) in place with the drug mixture (5) in contact with the vessel wall. The sheath (25) can be used as a conduit for fluid delivery or for device delivery. A sheath proximal portion (125) can be made porous to blood flow if desired to allow for blood perfusion through the sheath (25).

In another embodiment shown in FIG. 5 the drug mixture (5) is located on the inwardly facing surface (50) of the sheath (25) as described in FIGS. 4A and 4B. In this embodiment, the sheath distal end (70) extends out of the patient's body thereby giving the sheath (25) longer length of inverted wall (40). The device is used in a manner similar to that described in FIGS. 4A and 4B except that after retraction of the sheath (25) and following the inflation of the balloon (15), the sheath (25) can be withdrawn by retracting of the sheath distal end (70) in a proximal direction. This retraction method allows the sheath (25) to be removed from the lesion site using a tank tread motion that does not involve shearing of the drug mixture (5). Reference numerals used in the drawings have the same description as found in other figures.

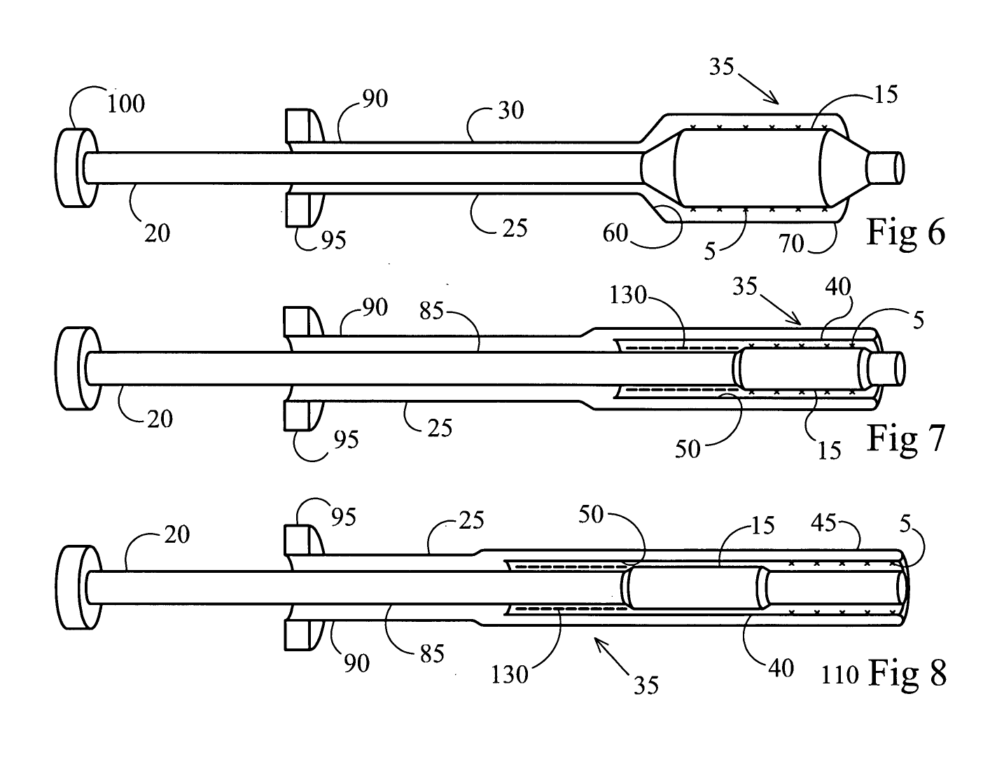

In one more embodiment shown in FIG. 6 a sheath (25) is shown where the sheath distal portion (35) is not inverted. The sheath (25) is a thin-walled tubular member (30) that extends over the balloon catheter (20) and over the balloon (15) that has a drug mixture (5) located on the outside surface (10) of the balloon (15). The sheath (25) covers the drug mixture (5) to protect it from exposure to blood and tissue while it is being delivered to the lesion site. Once it has reached its proper position with respect to the lesion, the sheath (25) is retracted proximally to expose the drug coated balloon (15) to the vessel wall. Inflation of the balloon (15) then places the drug into contact with the vascular lesion. A slip agent can be placed between the sheath (25) and the balloon catheter (20) to allow the sheath (25) to slide relative to the balloon catheter (20). Such slip agent can include silicone oil, hydrogels, Teflon coating, or other material that allows slippage to occur. The slip agent can be applied to either the balloon catheter (20) or the sheath inner surface (60). The sheath (25) also can help to hold the balloon (15) into a smaller profile during delivery. The sheath (25) can be formed from materials commonly used to form thin-walled dilatation balloons as mentioned earlier.

In a further embodiment shown in FIG. 7 a self-expanding stent (130) is located along the balloon catheter shaft (85) at a location proximal to the balloon (15) and is held from expansion by the inwardly-facing surface of the inverted wall (40) of the sheath (25). The drug mixture (5) is located on the balloon (15) and is covered by the sheath distal portion during delivery to the lesion site. The sheath (25) performs the functions of protecting the drug mixture (5) from exposure to the blood during delivery as well as providing a delivery sheath (25) for a self-expanding stent (130). Once the sheath (25) and balloon catheter (20) have reached the lesion site, the sheath proximal end (90) is retracted to expose the drug mixture (5) located on the balloon outside surface (10). The balloon (15) is inflated to place the drug mixture (5) into contact with the vessel wall. Proximal retraction of the sheath proximal end (90) causes the self-expanding stent (130) to be released into the blood vessel. The balloon (15) can be used to post-dilate the self-expanding stent (130) to place it into full contact with the lesion and ensure it is fully expanded.

In yet another embodiment shown in FIG. 8 the self-expanding stent (130) is located on the shaft (85) of the balloon catheter (20) proximal to the balloon (15). The inwardly-facing surface of the sheath distal portion (35) is holding the self-expanding stent (130) into its reduced diameter configuration for delivery to the lesion site. The drug mixture (5) is located on the sheath inwardly-facing surface (50). The sheath (25) serves to protect the drug mixture (5) from exposure to the blood and vessel wall during delivery and to provide a sheath (25) for delivery of the self-expanding stent (130). Proximal retraction of the sheath (25) causes the drug coated inwardly-facing surface (50) of the sheath (25) to be located on the sheath outer surface (45) and exposed to the vessel wall with the drug mixture (5) positioned over the balloon (15). The balloon (15) can be inflated to cause the balloon (15) and sheath (25) to expand and come into contact with the lesion site. Further retraction of the sheath (25) causes the self-expanding stent (130) to be released at the lesion site. Post-dilation of the stent can be performed using the balloon (15) if desired.

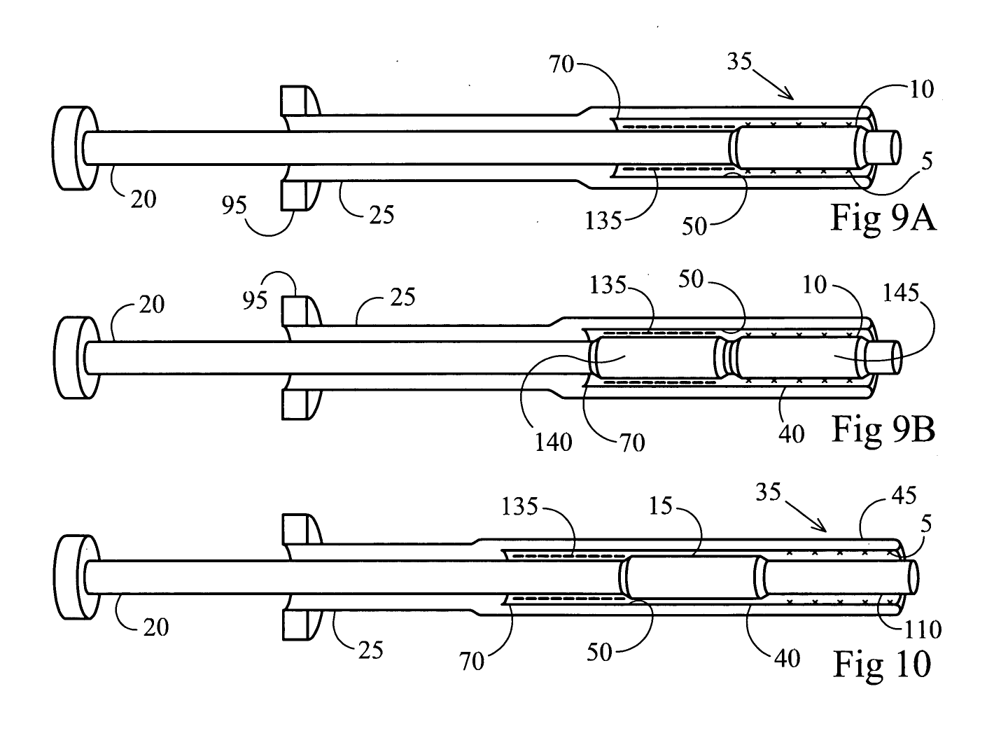

Further embodiments of the sheath (25) are shown in FIGS. 9A and 9B with the drug mixture (5) located on the balloon outside surface (10). The inverted wall (40) of the sheath (25) is located over the balloon catheter (20). A balloon-expandable stent (135) can be located on the shaft (85) of the balloon catheter (20) proximal to the balloon (15) as shown in FIG. 9A. The inwardly-facing surface (50) of the sheath distal portion (35) covers the drug mixture (5) and serves to hold the balloon-expandable stent (135) in a configuration that is a partially open configuration. The sheath (25) and balloon catheter (20) are delivered to the site of a lesion and the sheath (25) is retracted proximally to expose the drug coated balloon to the blood and vessel. The balloon (15) is expanded to place the drug mixture (5) into contact with the lesion site. The balloon catheter (20) is retracted proximally within the partially open balloon-expandable stent (135) and is expanded to lock the balloon (15) onto the stent. The sheath (25) is then retracted further proximally to expose the stent that is positioned onto the balloon outside surface (10) to the vessel. Positioning the stent at the lesion site and inflating the balloon (15) places the stent at the proper location to support the lesion site. To ensure that the balloon (15) can be retracted into the balloon-expandable stent (135), a proximal balloon portion (140) can be located proximal to the main balloon portion (145) as shown in FIG. 9B. The proximal balloon portion (140) is intended to partially inflate the stent to an intermediate configuration that ensures that the main balloon portion (145) can pass within it in a deflated configuration. During delivery the balloon-expandable stent (135) can be formed into a small diameter configuration. The proximal balloon portion (140) can also be a second balloon if desired.

In yet a further embodiment shown in FIG. 10 a sheath (25) that contains a drug mixture (5) also provides for delivery of a balloon-expandable stent (135). The stent can be located along the balloon catheter shaft (85) located proximal to the balloon. The drug mixture (5) is located on the inwardly-facing surface (50) of the sheath distal portion (35). The sheath (25) is located over the balloon catheter (20) and provides protection to the drug mixture (5) during delivery to the lesion site. Once the sheath (25) and balloon catheter (20) have reached the lesion site, the sheath (25) is retracted proximally to expose the drug mixture (5) located on the sheath (25) which has been brought into a position over the balloon (15) on the sheath outer surface (45) facing the lesion site. Expansion of the balloon (15) causes the balloon (15) and the sheath (25) to expand and place the drug into contact with the lesion. The balloon catheter (20) can be retracted proximally to place the balloon (15) into position within the stent. Partial dilation causes the balloon (15) to lock onto the stent. Proximal retraction of the sheath (25) then allows the stent loaded balloon (15) to be exposed to the blood vessel and the balloon (15) can then be inflated at the lesion site to place the stent.

The stent in this embodiment as well as all the embodiments described in FIGS. 7-10 can be bare metal stents. Bare metal stents will provide an improved healing response in comparison to most drug eluting stents. Late stent thrombosis shall be reduced by using the techniques described in these embodiments due to the improved healing of the bare metal stent which does not contain a drug which can inhibit healing. Also, the more continuous surface application of the drug mixture (5) via the balloon (15) or the sheath (25) surface will improve the restenosis rates over that seen by discontinuous delivery of drug via the struts of the drug eluting stents. The application of a drug mixture via a drug eluting balloon or sheath may also be delivered without the use of a bare metal stent and therefore at a lower cost than with the use of a stent.

1. A sheath that is able to provide improved function to an angioplasty balloon catheter, the balloon catheter having a balloon located on its distal segment that is deflated during delivery to a lesion site and that can be inflated to place a force onto a blood vessel wall, a drug mixture being located between the balloon and the vessel wall during balloon inflation for delivery into the vessel wall, said sheath comprising;
A. a flexible thin-walled tubular member having a proximal end, a distal end and a distal portion, said distal portion able to allow passage of the distal segment of the angioplasty balloon catheter therein,
B. said distal portion of said sheath having an inverted wall such that at least a portion of the outer surface becomes an inwardly-facing surface and at least a portion of the inner surface is contiguous with an isolated inner surface,
C. said distal portion of said sheath being placed over the distal segment of the balloon catheter such that the inwardly-facing surface of said sheath is placed adjacent to the distal segment of the balloon catheter to provide protection to the drug mixture against exposure to blood and tissue interaction during delivery to the lesion,
D. said proximal end being retractable to provide exposure of the drug mixture to the blood vessel wall at the site of the lesion without providing shearing motion to the drug mixture, thereby leaving the drug intact for delivery to the vessel wall during balloon inflation.

2. The sheath of claim 1 wherein at least a portion of said inverted distal portion of said sheath is located distal to the balloon during delivery.

3. The sheath of claim 1 wherein said distal end of said sheath is attached to the distal segment of the balloon catheter.

4. The sheath of claim 1 wherein said sheath has the drug attached to the inwardly-facing surface.

5. The sheath of claim 1 wherein said sheath covers the drug mixture which is attached to the balloon during delivery.

6. The sheath of claim 1 wherein said sheath is inverted along a substantial portion of its entire length.

7. The method of protecting a drug during the delivery of an angioplasty catheter and the drug to a lesion site located in a tubular member of the body comprising the steps;
A. placing a thin-walled tubular sheath having an inverted distal portion over the angioplasty catheter and over the drug mixture,
B. delivering the sheath containing the balloon catheter and drug to the site of the lesion, said sheath providing protection to the drug mixture against exposure to the blood and tissues,
C. withdrawing the sheath relative to the balloon catheter to expose the drug mixture without generating shear that could displace the drug, wherein the balloon catheter pushes the drug against the vessel wall as it is inflated.

8. The method of claim 7 further comprising the step of expanding the angioplasty catheter to push the drug against the tubular member of the body, and withdrawing said sheath and the angioplasty catheter from the tubular member of the body.

9. The method of claim 7 wherein the balloon catheter is held stationary as the sheath is withdrawn proximally.

10. The sheath of claim 7 wherein the sheath is held stationary as the balloon catheter is advanced distally.

11. A sheath that is able to provide improved function to an angioplasty balloon catheter, the balloon catheter having a balloon located on its distal segment that is deflated during delivery to a lesion site and that can be inflated to place a force onto a blood vessel wall, a drug mixture being located between the balloon and the vessel wall during balloon inflation for delivery into the vessel wall, and a stent located between the balloon catheter and said sheath, said sheath comprising;
A. a flexible thin-walled tubular member having a proximal end, a distal end and a distal portion, said distal portion able to allow passage of the distal segment of the angioplasty balloon catheter therein,
B. said distal portion of said sheath having an inverted wall such that at least a portion of the outer surface becomes an inwardly-facing surface and at least a portion of the inner surface becomes an isolated inner surface,
C. said distal portion of said sheath being placed over the distal segment of the balloon catheter such that the inwardly-facing surface of said sheath is placed adjacent to the distal segment of the balloon catheter to provide protection to the drug mixture against exposure to blood and tissue interaction during delivery to the lesion,
D. the inwardly-facing surface of said sheath being in contact with the stent during delivery of the sheath to the lesion,
E. said proximal end being retractable to provide exposure of the drug mixture and the stent to the blood vessel wall at the site of the lesion without providing shearing motion to the drug mixture, thereby leaving the drug intact for delivery to the vessel wall during balloon inflation.

12. The sheath of claim 11 wherein the stent is a self-expanding stent that is held by said inwardly facing surface; retraction of said proximal end of said sheath providing release of the stent into the blood vessel.

13. The sheath of claim 11 wherein the stent is a balloon expandable stent that is expanded by the balloon at the lesion site.
